(19) United States
(12) Patent Application Publication
(10) Pub. No.: US 2024/0050064 A1
(43) Pub. Date: Feb. 15, 2024

(54) Ultrasonic Surgical Holder

(71) Applicant: Astron Medtech Co., Ltd., Taipei City (TW)

(72) Inventors: Yi Hsi Huang, Taipei City (TW); I-Lin Tsai, Taipei City (TW); Kuei-Hua Chen, Taichung City (TW)

(21) Appl. No.: 18/343,362

(22) Filed: Jun. 28, 2023

Related U.S. Application Data
(60) Provisional application No. 63/396,681, filed on Aug. 10, 2022.

(51) Int. Cl. A61B 8/00

(52) U.S. Cl. CPC A61B 8/4209

(57) ABSTRACT

An ultrasonic surgical holder has an assembly portion connected to at least one operating portion having an end extending toward an axial direction. An operating space is formed between the assembly portion and the operating portion. The assembly portion is designed for detachable assembling with an ultrasonic probe. The operating portion features a detachable structure that includes a surgical device to aid the operator in positioning, marking, and determining the location of the surgical operation on the body part's surface during the subsequent operation.

### FIELD OF THE INVENTION

The present invention relates to a surgical holder, specifically an ultrasonic surgical holder.

### BACKGROUND OF THE INVENTION

With advancements in medicine, diagnostic tools for surgery are continually evolving. Ultrasonic instruments have become a popular choice for obtaining clear medical images during surgery, as they can penetrate and reflect into the body and organs for imaging. The equipment is simple, convenient, and non-invasive, making it the preferred choice for complementary diagnosis.

Surgeons use ultrasonic instruments to locate the surgical site inside the body from the outside, allowing them to perform surgery accurately, achieve the best surgical quality, and avoid unnecessary damage to other tissues.

However, during surgery, after positioning the ultrasonic instrument, surgeons need to use a marking tool to mark the surgical site on the body surface before using a new surgical device, resulting in the need for repeated instrument changes. This not only complicates the overall process but also wastes time. Therefore, there is a pressing need in the industry for a composite holder with interchangeable surgical instruments that can facilitate positioning, marking, and performing surgery.

### SUMMARY OF THE INVENTION

To develop a composite holder with interchangeable surgical instruments and achieve convenient positioning, marking, and performing surgery. The present invention provides an ultrasonic surgical holder comprising an assembly portion that is at least partially connected to at least one operating portion having an end extending in the direction of an axis, an operating space between the assembly portion and the operating portion. The assembly portion is used for detachable assembly with an ultrasonic probe such that the irradiating end of the ultrasonic probe corresponds to the operating space and forms an irradiating plane extending along the axial direction.

Wherein the operating portion comprises at least one module for detachable assembly with a surgical device and a module assembly structure for detachably assembling at least one module. The operating end of the surgical device corresponds horizontally to the irradiating plane when the module is assembled with the surgical device and disposed in the module assembly structure.

Wherein the operating portion is pivoted relative to the assembly portion and away from the irradiating plane.

When the ultrasonic surgical holder is used, it can be assembled with the ultrasonic probe through the assembly portion and irradiate the body part located in the operating space so that one of the cross-sections of the body part corresponds to the irradiating plane, and the image of the cross-section can be formed. The operator can confirm the position of the surgical site and the depth relative to the surface of the body part according to the cross-section.

The ultrasonic surgical holder provided by the present invention can be assembled with the ultrasonic probe and surgical devices (such as laser markers or surgical needles) at the same time to set up the surgical devices according to the depth of the surgical site. The operator can position, mark, and determine the surgical position on the surface of the body part during the subsequent operation.

### BRIEF DESCRIPTION OF THE DRAWINGS

FIG. 1 is a schematic view of the first preferred embodiment of the present invention;

FIG. 2 is a schematic view of the second preferred embodiment of the present invention;

FIG. 3 is an exploded view of the second preferred embodiment of the present invention;

FIG. 4 is a schematic view of the first step of the second preferred embodiment of the present invention; and

FIG. 5 is a schematic view of the second step of the second preferred embodiment of the present invention.

### DESCRIPTION OF THE PREFERRED EMBODIMENTS

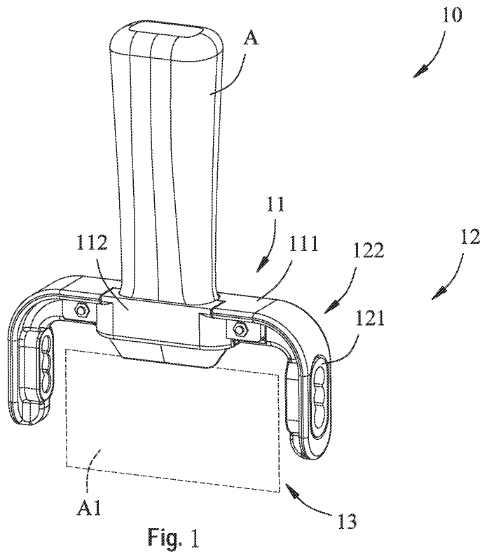

Referring to FIGS. 1 to 3, the ultrasonic surgical holder 10 provided by the present invention comprises an assembly portion 11 and at least one operating portion 12. The operating portion 12 is connected to at least a part of the assembly portion 11 where its end extends in the direction of an axis. An operating space 13 is formed between the assembly portion 11 and the operating portion 12 to accommodate a body part in the operating space 13.

The assembly portion 11 is used for detachable assembly connection with an ultrasonic probe A so that the irradiating end of the ultrasonic probe A corresponds to the operating space 13. When the ultrasonic probe A is irradiated to the operating space 13, an irradiating plane A1 extending along the axial direction is formed.

Wherein the method of assembling the assembly portion 11 and the ultrasonic probe A is not limited. The assembly portion 11 may be a fixture for holding the ultrasonic probe A, or it may mutually snap with at least a part of the ultrasonic probe A. In this embodiment, the assembly portion 11 is rod-like and includes a first assembly 111 and a second assembly 112. A first recess 113 is recessed at the central portion of the side of the first assembly 111, and a second recess 114 is recessed at the central portion of the side of the second assembly 112. Both ends of the first assembly 111 can be screwed with both ends of the second assembly 112 by screws and nuts to form the assembly portion 11.

The first recess 113 and the second recess 114 are set correspondingly to each other after the first assembly 111 and the second assembly 112 are assembled. A perforation is formed through the assembly portion 11 in the axial direction, corresponding to at least a part of the outer diameter of the ultrasonic probe A, so that the ultrasonic probe A can be stabilized in the perforation after being assembled with the first assembly 111 and the second assembly 112.

In this way, when the ultrasonic surgical holder is used, it can be assembled with the ultrasonic probe A through the assembly portion 11 and irradiate the body part located in the operating space 13. This allows one of the cross-sections of the body part to correspond to the irradiating plane A1, and the image of the cross-section can be formed. This enables an operator to confirm the position of the surgical site and the depth relative to the surface of the body part according to the cross-section.

The operating portion 12 comprises at least one module 121 and a module assembly structure 122 for detachable assembly of the at least one module 121 therein. The module 121 is detachably assembled with a surgical device. When the module 121 is assembled with the surgical device and disposed in the module assembly structure 122, the operating end of the surgical device corresponds horizontally to the irradiating plane A1.

Preferably, the module 121 can displace the surgical device along the axial direction. This allows the operating end of the surgical device to correspond to the irradiating plane A1 horizontally along the axial direction. In this way, the operator can assemble the corresponding module 121 on the module assembly structure 122 according to the surgical device to be used.

In this embodiment, two operating portions 12 are provided at both ends of the assembly portion 11 and extend in the direction of the axis. The operating space 13 is formed between the assembly portion 11 and the two operating portions 12. The module assembly structure 122 includes an assembly perforation 1212 corresponding to the operating space 13 and penetrating through the operating portion 12.

At least a portion of module 121 can be detachably assembled to the inner diameter of assembly perforation 1212, and there are various methods to assemble the module 121 to each surgical device. The module 121 can be a fixture for each surgical device or designed for one surgical device and mutually snap with at least a portion of the surgical device.

In the first embodiment for the operating portion 12 provided by the present invention, when the surgical device is a laser calibrator B1, the module 121 is a block that corresponds to the inner diameter of the assembly perforation 1212. The module 121 is arranged along the axial direction to penetrate at least one holding hole 1211 parallel to the irradiating plane A1, and the inner diameter of the holding hole 1211 corresponds to the outer diameter of the laser calibrator B1. In this way, the laser calibrator B1 can be set in one of the holding holes 1211 according to the depth of the surgical site, and the laser end of the laser calibrator B1 can be used to make a mark on the surface of the body part to facilitate the operator in determining the operation position during the subsequent operation.

In the second embodiment for the operating portion 12 provided by the present invention, when the surgical device is a surgical needle B2, different from the aforementioned embodiment, the module 121 includes a needle holding member 1212 in the form of a hollow rod. The module 121 is arranged along the axis direction to penetrate at least one needle holding hole 1213 parallel to the irradiating plane A1, and the inner diameter of the needle holding hole 1213 corresponds to the outer diameter of the needle holding member 1212. The inner diameter of the needle holding member 1212 is slightly larger than that of the surgical needle B2 so that the surgical needle B2 can be displaced in the needle holding member 1212. In this way, the operator can use the module 121 to guide the surgical needle B2 into the body part by accurately matching the depth of the surgical site (in particular, by matching the marking of the laser calibrator B1 on the surface of the body part).

Since the surgical needle B2 is a slender needle, at least a portion of the surgical needle B2 remains in the needle holding member 1212 when it is inserted into the body part and the surgical site. To facilitate the detachment of the surgical needle B2 from the ultrasonic surgical holder 10 so that the operator can perform the subsequent surgical procedure, the edge of the operating portion 12 is pierced with a first rejection notch 123 toward the operating space 13, and the first rejection notch 123 is connected to the assembly perforation 12. The module 121 is pierced with a second rejection notch 124 corresponding to the first rejection notch 123 toward the operating space 13, and the second rejection notch 124 is connected to the first rejection notch 123 and the needle holding hole 1213 when the module is assembled with the module assembly structure.

This enables the operator to detach the ultrasonic surgical holder 10 from the surgical needle B2 that is inserted into the body part. The needle holding member 1212 can be removed from the needle holding hole 1213 relative to the operating space 13, leaving at least a section of the surgical needle B2 in the hole 1213. To move the ultrasonic surgical holder 10 away from the body part, the surgical needle B2 is displaced along the needle holding hole 1213, the second rejection notch 124, and the first rejection notch 123, making it easy to detach the surgical needle B2 from the ultrasonic surgical holder 10.

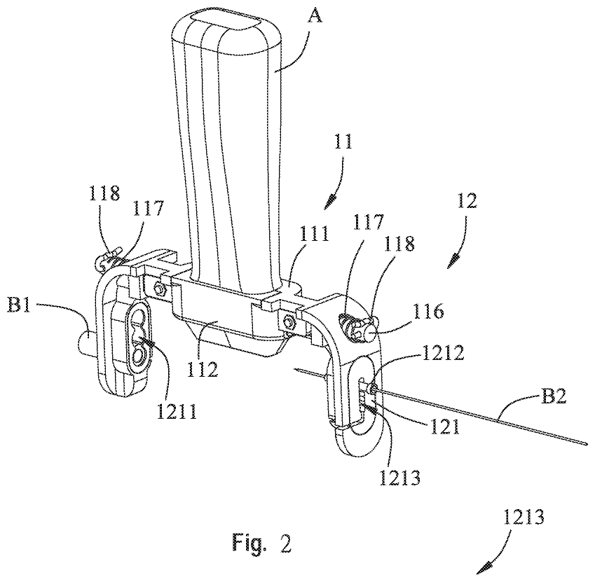

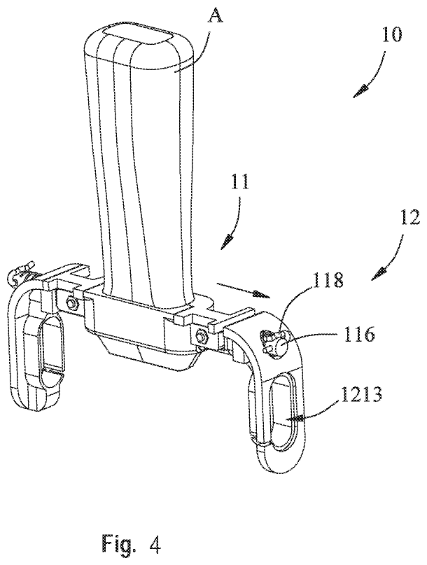

The present invention further provides a second preferred embodiment of the ultrasonic surgical holder 10, as shown in FIGS. 2 to 5. In this embodiment, the operating portion 12 can pivot relative to the assembly portion 11 and away from the irradiating plane A1, allowing the operator to move the operating portion 12 as required to avoid interference when using the ultrasonic probe A. The direction in which the operating portion 12 pivots with respect to the assembly portion 11 is not limited and can be outwardly or radially with respect to the irradiating plane A1.

In this embodiment, the operating portion 12 can be interlocked or pivoted with the assembly portion 11 as desired. The assembly portion 11 has a projection 115 with a regular polygon cross-section at both ends, and the end face of the projection 115 has a rotating shaft 116 projecting along the longitudinal direction. The minimum diameter of the cross-section of the projection 115 is greater than or equal to the maximum diameter of the cross-section of the rotating shaft 116. Wherein the regular polygon is a regular quadrilateral.

An assembly recess 125 corresponding to the projection 115 is recessed at the connecting position of the operating portion 12 and the assembly portion 11, and a rotating shaft hole 126 corresponding to the rotating shaft 116 is pierced through the assembly recess 125. A spring 117 is provided between the assembly portion 11 and the operating portion 12. When the assembly portion 11 is assembled with the operating portion 12, the rotating shaft 116 can be rotatably pierced in the rotating shaft hole 126, and the projection 115 can be selectively positioned in the assembly recess 125.

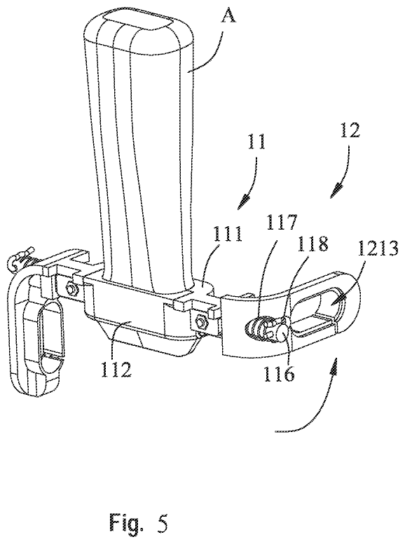

Preferably, the end of the rotating shaft 116 is provided with a resting member 118, and the spring 117 is a compression spring which is sleeved at the end of the rotating shaft 116 and between the operating portion 12 and the resting member 118. In the locked state (as shown in FIG. 2 and FIG. 5), the spring 117 against between the assembly portion 11 and the operating portion 12 urges the projection 115 to a position in the assembly recess 125, so the operating portion 12 cannot be pivoted against the assembly portion 11 due to the regular polygonal assembling relationship between the projection 115 and the assembly recess 125.

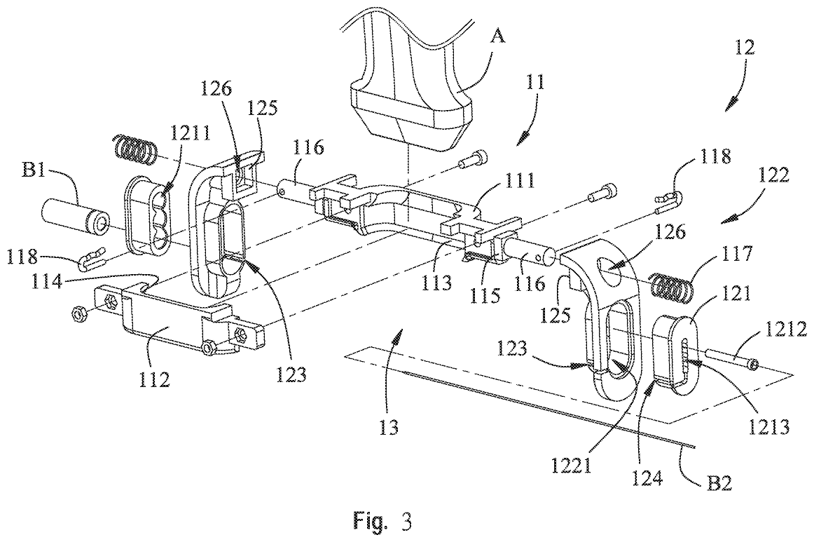

When rotation of the operating portion 12 is required, the operating portion 12 is pulled away from the assembly portion 11 (as shown in FIG. 3), and the spring 117 is compressed, causing the projection 115 to separate from the assembly recess 125. The operating portion 12 can then be rotated relative to the assembly portion 11 through the rotating shaft 116 and the rotating shaft hole 126, while the projection 115 is angularly misaligned with the assembly recess 125.

When the operating portion 12 is rotated with respect to the assembly portion 11 until the shape of the projection 115 corresponds again to the assembly recess 125, the spring 117 pushes the projection 115 with its elastic recovery force. This causes the projection to be displaced into the assembly recess 125 and return to the locked state. For example, in this embodiment, the regular polygon is a regular quadrilateral. When the operating portion 12 is rotated 90 degrees with respect to the assembly portion 11, the projection 115 corresponds to the shape of the assembly recess 125, and the spring 117 can push the projection 115 to be displaced into the assembly recess 125.

The present invention provides the ultrasonic surgical holder 10, which can assemble the ultrasonic probe A and the surgical devices simultaneously. This makes it easy to position, mark, and perform the surgery during the procedure.

What is claimed is:

1. An ultrasonic surgical holder comprising an assembly portion, at least a part of which is connected to at least one operating portion having an end extending in the direction of an axis, and an operating space is formed between the assembly portion and the operating portion, wherein the assembly portion is detachably assembled with an ultrasonic probe so that the irradiating end of the ultrasonic probe corresponds to the operating space and forms an irradiating plane extending along the axial direction.

2. The ultrasonic surgical holder according to claim 1, the operating portion comprises at least one module that is detachably assembled with surgical devices and a module assembly structure for detachably assembling at least one module, wherein, when the module is assembled with the surgical device and disposed in the module assembly structure, the operating end of the surgical device corresponds horizontally to the irradiating plane.

3. The ultrasonic surgical holder according to claim 2, the assembly portion is rod-like and includes a first assembly and a second assembly, wherein a first recess is recessed at the central portion of the side of the first assembly, and a second recess is recessed at the central portion of the side of the second assembly, wherein the both ends of the first assembly and the both ends of the second assembly are assembled with each other, and the first recess and the second recess are set correspondingly to each other to form a perforation through the assembly portion in the axial direction, and the perforation corresponds to at least a part of the outer diameter of the ultrasonic probe.

4. The ultrasonic surgical holder according to claim 3, two operating portions are provided at the both ends of the assembly portion, and the module assembly structure includes an assembly perforation corresponding to the operating space and penetrating through the operating portion.

5. The ultrasonic surgical holder according to claim 4, the module is a block and is formed corresponding to the inner diameter of the assembly perforation, the module being arranged along the axial direction to penetrate at least one holding hole parallel to the irradiating plane for holding a laser calibrator.

6. The ultrasonic surgical holder according to claim 4, the module is a block and is formed corresponding to the inner diameter of the assembly perforation, the module being arranged along the axial direction to penetrate at least one needle holding hole parallel to the irradiating plane for holding a needle holding member in the form of a hollow rod, which is used to guide the penetration of a surgical needle into the corresponding body part.

7. The ultrasonic surgical holder according to claim 2, the edge of the operating portion is pierced with a first rejection notch toward the operating space, and the first rejection notch is connected to the assembly perforation, and the module is pierced with a second rejection notch corresponding to the first rejection notch toward the operating space, when the module is assembled with the module assembly structure, the second rejection notch is connected to the first rejection notch and the needle holding hole.

8. The ultrasonic surgical holder according to claim 1, the operating portion is pivoted relative to the assembly portion and away from the irradiating plane.

9. The ultrasonic surgical holder according to claim 8, the assembly portion has a projection having a regular polygon cross section along a longitudinal direction at both ends of the assembly portion, and the end face of the projection has a rotating shaft projected along the longitudinal direction, the minimum diameter of the cross-section of the projection is greater than or equal to the maximum diameter of the cross-section of the rotating shaft;
an assembly recess corresponding to the projection is recessed at the connecting position of the operating portion and the assembly portion, and a rotating shaft hole corresponding to the rotating shaft is pierced through the assembly recess, the rotating shaft is rotatably pierced in the rotating shaft hole; and
a spring is a compression spring placed between the assembly portion and the operating portion, wherein,
when the assembly portion is assembled with the operating portion, the spring urges the projection to a position in the assembly recess; and
when the spring is compressed, the projection is separated from the assembly recess so that the rotating shaft rotates relative to the rotating shaft hole, and the projection is angularly misaligned with the assembly recess.

10. The ultrasonic surgical holder according to claim 8, wherein the regular polygon is a regular quadrilateral.

11. The ultrasonic surgical holder according to claim 10, the end of the rotating shaft is provided with a resting member, and the spring is sleeved at the end of the rotating shaft and placed between the operating portion and the resting member.
